(19) United States
(12) Patent Application Publication
(10) Pub. No.: US 2006/0173397 A1
(43) Pub. Date: Aug. 3, 2006

(54) Ophthalmology implants and methods of manufacture

(76) Inventors: Hosheng Tu, Newport Coast, CA (US); Jianbo Zhou, Rancho Santa Margarita, CA (US); David Haffner, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/286,779

(22) Filed: Nov. 22, 2005

Related U.S. Application Data
(60) Provisional application No. 60/631,294, filed on Nov. 23, 2004.

(51) Int. Cl. A61M 5/00

(52) U.S. Cl. 604/8

(57) ABSTRACT

The present disclosure provides an ophthalmology implant and methods for treating glaucoma or optic neural transmission deficiency, wherein at least a portion of the implant is made of or includes a nanometer-sized substance, such as nanotubes, nanofibers, sheets from nanotubes, nanowires, nanofibrous mesh and the like.

### RELATED APPLICATIONS

The present application claims priority from U.S. Provisional Application No. 60/631,294, filed Nov. 23, 2004, entitled “Ophthalmology Implants and Methods of Manufacture,” the entirety of which is incorporated herein by reference.

### FIELD OF THE INVENTIONS

This disclosure relates to medical devices made of or incorporated with nanometer-sized substances. More particularly, this disclosure relates to ophthalmology implants and processes of manufacture thereof for treating glaucoma and related eye illness.

### BACKGROUND OF THE INVENTIONS

Nanotechnology is the creation, manipulation, and manufacture of compounds and devices so small they are measured in nanometers, with one nanometer equaling one-billionth of a meter. By convention, nanotechnology usually refers to things that are 100 nanometers or less in size. Helped along by the advent of powerful microscopes that allowed scientists to observe things on a molecular level, a scanning transmission electron microscope with a resolution of less than one angstrom has been developed. Accordingly, a non-nanometer-sized medical device for ophthalmology use may be made of many nanometer-sized substances or include at least a portion of the device made of nanometer-sized substances. The ability to manipulate individual atoms and molecules would lead to new materials with entirely new properties, which in turn can be used as building blocks for new products and increasingly complex systems.

One of the first innovations in the field of nanotechnology was the advent of carbon nanotubes. Carbon nanotubes are small sheets of a carbon lattice or graphite, rolled into single-wall tubes with an average diameter of about 1.2 to 1.4 nanometers, or multi-wall tubes with diameters of about 10 to 300 nanometers. Their lengths range from 1 mm to 20 cm. The carbon nanotubes may be good conductors of electricity. They are one of the strongest materials known with a stretchability of up to 30% of their original length.

Some prior art discloses a device incorporating nanotubes into nerve cells with the goal of making the prosthetic device that people can control as they would intact limbs. The nanotubes can be woven into carbon nanofibers that may eventually be used to make neural and orthopedic implants that are more durable and more compatible with human tissue than current implant. It is contemplated that the carbon nanotubes are made of carbon that is categorized organic by definition, the risk of scar tissue forming around them could be less than when other materials, such as silicon, are introduced into the body. A renal dialysis button made of BIOCARBON® (Bentley Laboratories, Irvine) several years ago demonstrated the biocompatibility of a pure carbon device clinically.

Nanomedicine is the medical application of nanotechnology and related research. It covers areas such as nanoparticle drug delivery, nanometer-sized medical devices, implants with at least one nanometer-sized dimension, devices incorporated with nanometer-sized substances, and possible future applications of molecular nanotechnology.

For ophthalmology applications with nanomedicine, glaucoma and optical nerve therapy may constitute two major fields of interest. As is well known in the art, a human eye is a specialized sensory organ capable of light reception and is able to receive visual images. Aqueous humor is a transparent liquid that fills the region between the cornea, at the front of the eye, and the lens. A trabecular meshwork, located in an anterior chamber angle formed between the iris and the cornea, serves as a drainage channel for aqueous humor from the anterior chamber, which maintains a balanced pressure within the anterior chamber of the eye.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (hereinafter referred to as “aqueous”) to enter Schlemm's canal, which then empties into aqueous collector channels in the posterior wall of Schlemm's canal and then into aqueous veins, which form the episcleral venous system. Aqueous is continuously secreted by a ciliary body around the lens, so there is a constant flow of aqueous from the ciliary body to the anterior chamber of the eye. Pressure within the eye is determined by a balance between the production of aqueous and its exit through the trabecular meshwork (major route) and uveal scleral outflow (minor route). The portion of the trabecular meshwork adjacent to Schlemm's canal (the juxtacanilicular meshwork) causes most of the resistance to aqueous outflow.

Glaucoma is broadly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye. Open-angle glaucoma is any glaucoma in which the exit of aqueous through the trabecular meshwork is diminished while the angle of the anterior chamber remains open. For most cases of open-angle glaucoma, the exact cause of diminished filtration is unknown. Primary open-angle glaucoma is the most common of the glaucomas, and is often asymptomatic in the early to moderately advanced stages of glaucoma. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

All current therapies for glaucoma are directed toward decreasing intraocular pressure. Currently recognized categories of drug therapy for glaucoma include: (1) Miotics (e.g., pilocarpine, carbachol, and acetylcholinesterase inhibitors), (2) Sympathomimetics (e.g., epinephrine and dipivalylepinephxine), (3) Beta-blockers (e.g., betaxolol, levobunolol and timolol), (4) Carbonic anhydrase inhibitors (e.g., acetazolamide, methazolamide and ethoxzolamide), and (5) Prostaglandins (e.g., metabolite derivatives of arachindonic acid). Medical therapy includes topical ophthalmic drops or oral medications that reduce the production of aqueous or increase the outflow of aqueous. However, drug therapies for glaucoma are sometimes associated with significant side effects. The most frequent and perhaps most serious drawback to drug therapy is that patients, especially the elderly, often fail to correctly self-medicate. Such patients forget to take their medication at the appropriate times or else administer eye drops improperly, resulting in under- or over-dosing. Because the effects of glaucoma are irreversible, when patients dose improperly, allowing ocular concentrations to drop below appropriate therapeutic levels, further permanent damage to vision occurs. Furthermore, current drug therapies are targeted to be deposited directly into the ciliary body where the aqueous is produced. In addition, current therapies do not provide for a continuous slow-release of the drug. When drug therapy fails, surgical therapy is pursued.

Surgical therapy for open-angle glaucoma consists of laser trabeculoplasty, trabeculectomy, and implantation of aqueous stents after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if ocular morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure). For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Q-switched Neodynium (Nd) YAG lasers also have been investigated as an optically invasive trabeculopuncture technique for creating full-thickness holes in trabecular meshwork. However, the relatively small hole created by this trabeculopuncture technique exhibits a filling-in effect and fails.

Goniophotoablation is disclosed by Berlin in U.S. Pat. No. 4,846,172 and involves the use of an excimer laser to treat glaucoma by ablating the trabecular meshwork. This method did not succeed in a clinical trial. Hill et al. used an Erbium YAG laser to create full-thickness holes through trabecular meshwork (Hill et al., Lasers in Surgery and Medicine 11:341-346, 1991). This laser trabecular ablation technique was investigated in a primate model and a limited human clinical trial at the University of California, Irvine. Although ocular morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage is an “ab intemo” (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanulostomy (VC) and non penetrating trabeculectomy (NPT) are two new variations of filtering surgery. These are “ab extemo” (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous through the surgical opening will continue. The risk of placing a glaucoma drainage device also includes hemorrhage, infection, and diplopia (double vision).

Examples of implantable stents and surgical methods for maintaining an opening for the release of aqueous from the anterior chamber of the eye to the sclera or space beneath the conjunctiva have been disclosed in, for example, Hsia et al., U.S. Pat. No. 6,059,772 and Baerveldt, U.S. Pat. No. 6,050,970, both of which are incorporated herein by reference.

All of the above embodiments and variations thereof have numerous disadvantages and moderate success rates. They involve substantial trauma to the eye and require great surgical skill in creating a hole through the full thickness of the sclera into the subconjunctival space. The procedures are generally performed in an operating room and involve a prolonged recovery time for vision. The complications of existing filtration surgery have prompted ophthalmic surgeons to find other approaches to lowering intraocular pressure.

Because the trabecular meshwork and juxtacanilicular tissue together provide the majority of resistance to the outflow of aqueous, they are logical targets for surgical removal in the treatment of open-angle glaucoma. In addition, minimal amounts of tissue need be altered and existing physiologic outflow pathways can be utilized.

As reported in Arch. Ophthalm. (2000) 118:412, glaucoma remains a leading cause of blindness, and filtration surgery remains an effective, important option in controlling glaucoma. However, modifying existing filtering surgery techniques in any profound way to increase their effectiveness appears to have reached a dead end. The article further states that the time has come to search for new surgical approaches that may provide better and safer care for patients with glaucoma.

### SUMMARY OF THE INVENTION

It has been realized that what is needed in glaucoma management is a site-specific treatment method for placing a trabecular implant comprising at least a portion of nanometer-sized substance into Schlemm's canal for diverting aqueous humor from the anterior chamber into Schlemm's canal. In some aspects of the present disclosure, there is provided a method for optic neural management comprising implanting a conductive device having at least a portion of the device consisting of nanometer-sized substances configured to enhance electric conductance of the optical neural system for neural signal transmission.

A device and methods are provided for improved treatment of elevated intraocular pressure due to glaucoma. An implant is adapted for implantation within a Schlemm's canal or trabecular meshwork of an eye such that aqueous humor flows controllably from an anterior chamber of the eye to Schlemm's canal, bypassing the trabecular meshwork. In one embodiment, at least a portion of the implant comprises nanometer-sized substance or substrate, is configured of a nanostructure or is made of a nanosynthesis process effective in treating glaucoma or other ophthalmology indications. Further, depending upon the specific treatment contemplated, therapeutic agents (such as pharmaceuticals, genes, cells, proteins, anti-glaucoma agents, and/or growth factors) may be utilized in conjunction with the implant having nanometer-sized substance or nanostructure configuration. For example, U.S. application Ser. No. 10/706,300, filed Nov. 12, 2003, the entire contents of which is incorporated herein by reference, discloses several therapeutic agents that can be applied to an implant. Placement of the implant within the eye and incorporation, and eventual release, of a proven therapeutic therapy can slow the effects of glaucoma or treat other eye illness. In a further embodiment, the nanostructure of the device may be shaped like a sphere with a central hollow portion of the structure contemplated for holding therapeutic agents and functioning as devices to deliver therapeutic agents to specific disease sites in the body in a controlled manner.

In one aspect of the disclosure, an implant includes a nanometer-sized substance or substrate, a nanostructure configuration, or in which an implant is made of a nanosynthesis process is provided. In one embodiment, an implant includes a nanometer-sized substance or substrate, a nanostructure configuration, or is made of a nanosynthesis process that is implantable within a body channel. In another embodiment, the implant is loaded with a therapeutic agent effective in treating the tissue, which is controllably released from the device into tissue of the body channel.

Some aspects of the disclosure relate to a trabecular stent including at least a portion of the trabecular stent with nanometer-sized nanotubes (such as carbon nanotubes, carbon nanofibers, single-wall tubes or multi-wall tubes made of carbon nanotubes) configured in essentially parallel to aqueous flow direction inside the lumen of the stent. In another embodiment, at least a portion of the trabecular stent comprises nanometer-sized substance, such as nanotubes, nanofibers, nanowires, nanofibrous mesh, single-wall tubes or multi-wall tubes made of carbon nanotube sheets, and combinations thereof.

Some aspects of the disclosure relate to an implant including some nanometer-sized substance or substrate, a nanostructure configuration or being made of a nanosynthesis process, wherein the implant has adjustable anchoring capability. In a further embodiment, the implant is a trabecular stent that has adjustable anchoring capability within Schlemm's canal.

In another aspect of the disclosure, a method of implanting an implant within an eye is provided, comprising creating an incision through a conjunctival tissue at a limbus; radially incising an junction between an angle tissue and sclera, which is surgically extended until Schlemm's canal is entered posteriorly; and placing the implant within Schlemm's canal, wherein the implant resides and is adapted suitably for retention within the canal.

Some aspects of the disclosure relate to a method for optic neural management comprising implanting a conductive device having at least a portion of nanometer-sized substance configured to enable or enhance electric conductance of the optical neural system for neural signal transmission. In a further embodiment, the nanometer-sized substance comprises carbon nanotubes that are configured to be electrically conductive. In one embodiment, the implanting step is carried out at about the retina site. In one embodiment, the implanting step is carried out at about the optic nerve site. In another embodiment, the implanting step is carried out at about the optic disk site.

Some aspects of the present disclosure relate to a method of manufacturing an ophthalmology implant comprising coating at least a portion of the implant with a nanometer-sized substance or substrate configured for modifying the physical properties or surface properties of the implant. Some aspects of the disclosure are to provide a trabecular shunt or a tubular medical device sized and configured with a wall thickness less than about 100 nanometers, preferably less than about 10 nanometers, more preferably less than about 2 nanometers. In another embodiment, a ratio of wall thickness to inside diameter of a trabecular stent or a tubular medical device is less than about one-hundredth, preferably less than about one-thousandth, more preferably less than about one-ten thousandth, and most preferably less than about one-millionth.

Some aspects of the disclosure relate to an intraocular pressure sensor comprising nanosensor elements that are sized and configured less than 100 nanometers in at least one dimension. In one embodiment, the intraocular pressure sensor of the disclosure is a stand-alone device that monitors the intraocular pressure from within the anterior chamber (such as the one attached to a contact lens or intraocular lens) or from outside the eye (such as the one installed on a pair of glasses). In another embodiment, the intraocular pressure sensor of the disclosure is a part of the trabecular stent or the ophthalmology implant system, having the capability of sensing the IOP for monitoring and/or controlling the IOP.

In one embodiment, an implant for treating glaucoma is described, in which the implant includes an inlet portion having an inlet lumen extending therethrough. The inlet lumen is preferably in fluid communication with the anterior chamber of an eye when the implant is positioned within the trabecular meshwork of the eye. The implant also includes an outlet portion having an outlet lumen extending therethrough, the outlet lumen being in fluid communication with the inlet lumen and with an aqueous outflow channel of the eye when the outlet portion is disposed within the aqueous outflow channel of the eye and thereby permitting the transfer of fluid from the anterior chamber into the aqueous outflow channel. At least a portion of the implant preferably includes a nanostructure. The aqueous outflow channels can include at least one of Schlemm's canal, a collector channel, and an episcleral vein.

In one embodiment, an implant for treating glaucoma is described. The implant includes an inlet portion having an inlet that is configured to receive fluid from the anterior chamber of an eye when the implant is positioned within the trabecular meshwork of the eye. The implant also includes an outlet portion having an outlet that is configured to conduct fluid from the anterior chamber into at least one of Schlemm's canal, a collector channel, or an episcleral vein of the eye when the outlet portion is positioned within the eye, thereby permitting the transfer of fluid from the anterior chamber into Schlemm's canal, collector channels, or episcleral veins and a layer of nanotubes disposed in or on the implant.

### BRIEF DESCRIPTION OF THE DRAWINGS

FIG. 1 shows a coronal, cross-sectional view of an eye.

FIG. 2 shows a cross-sectional view of the anterior chamber angle of an eye.

FIG. 3 shows an embodiment of the trabecular implant constructed according to the principles of the disclosure.

FIG. 4 shows a front cross-sectional view of section 1-1 of FIG. 3, comprising a plurality of nanotubes.

FIG. 5 shows another embodiment of the trabecular implant constructed in accordance with the principles of the disclosure.

FIG. 6 shows a perspective view of the anterior chamber of an eye, illustrating the glaucoma device of the present disclosure positioned within the trabecular meshwork.

FIG. 7 illustrates a method of placement of the trabecular implant in an eye in accordance with the present disclosure.

FIG. 8A illustrates one configuration of a trabecular implant with at least a portion of the implant comprising nanometer-sized substances.

FIG. 8B illustrates the trabecular implant of FIG. 8A with adjustable anchoring capability.

FIG. 8C illustrates another configuration of a trabecular implant with at least a portion of the implant comprising nanometer-sized substances.

FIG. 8D illustrates the trabecular implant of FIG. 8C with adjustable anchoring capability.

### DETAILED DESCRIPTION OF THE EXEMPLARY EMBODIMENTS

Some exemplary embodiments of the disclosure described below relate particularly to surgical and therapeutic treatment of glaucoma through reduction of intraocular pressure. Some further exemplary embodiments relate to an ophthalmology nerve prosthesis comprising carbon nanotubes sized and configured for transmitting neural signals from light receptors to the brain. In one embodiment, the carbon nanotubes (woven or non-woven) are configured as carbon nanofibers and nanofibrous mesh. As used herein, nanofibrous mesh is intended to include, without limitation, a fibrous nanomesh. While the description sets forth various embodiment-specific details, it will be appreciated that the description is illustrative only and should not be construed in any way as limiting the disclosure. Furthermore, various applications of the disclosure, and modifications thereto, which may occur to those who are skilled in the art, are also encompassed by the general concepts described below.

One potential application of nanotechnology lies in nanosynthesis of materials (such as metal, plastic, biological or hydrogel) for the manufacture of more biocompatible medical implants. The use of metal nanosynthesis technology to manufacture coronary stents can reduce or eliminate surface abnormalities and irregularities, to minimize the presence of contaminants such as nickel, chromium and aluminum that can cause allergic and thrombogenic reactions, to improve fracture resistance and elastic properties, and to reduce overall stent metal volume. In one embodiment, development of nanomaterial provides new structures with exceptional strength as synthetic muscles for use in device actuators.

“Resin-gas injection-assisted bonding” is a technique used to bond nanosized components configured as a useable medical device. In one example, the device consisted of a 100-mm-wide channel with a fluid reservoir at each end. The device components were molded in two pieces, including a bottom platform containing the channel and reservoirs and a lid. After both parts were coated with a few drops of hydroxyethyl methacrylate (HEMA), they were fitted together. A short burst of nitrogen gas was then blown in one end of the device and out the other. This forced the adhesive to coat the inner surfaces on its way out. Finally, the entire device was cured using UV light. In one aspect of the disclosure, a nanosynthesis process to make an implant comprising a nanometer-sized substance or substrate utilizes the resin-gas injection-assisted bonding technique.

A medical-grade stainless steel that combines exceptional levels of strength with ductility may comprise nanometer-sized particles and impart a range of unique properties such as tensile strength, corrosion resistance, formability, and so forth. The particles are formed by means of a heat treatment process, whereas a novel phenomenon occurs within the material during heat treatment that produces nanoscale precipitates of a quasicrystalline structure. The results of in vitro testing show no cytotoxic potential and meets global standards related to allergies and skin irritations. Wire, tube, bar, strip, and rod shapes are available from this process.

The single-wall or multi-wall carbon nanotubes have been designed for use as dispersion additives in polyurethanes. Carbon nanotubes are extended buckminsterfullerene molecules, or “buckyballs,” spherical molecules constructed solely from 60 carbon atoms. The molecular structure provides exceptional strength. Some aspects of the disclosure provides an ophthalmology implant using medical grade material or polymers (such as polyurethane or silicone) with dispersed carbon nanotubes for enhancing strength of the implant.

Engineers at the University of California (UC), Berkeley, reported some progress to grow silicon nanowires and carbon nanotubes directly on microstructures at room temperature. One method of fabricating an ophthalmology device, exemplarily a trabecular stent, includes growing silicone nanowires or carbon nanotubes directly on a perform enabling the device to penetrate into eye tissue for fixing the device in place. Prior processes include producing nanomaterials separately and then manually connected to larger systems. Growing the nanotubes and nanowires onto microstructures eliminates the cumbersome steps involved in connecting them onto microstructures. It is contemplated that a trabecular stent having grown nanowires or nanotubes is sized and configured for anchoring the stent securely in place.

In a separate technology, a method is used to weave single-wall carbon nanotubes (SWNTs) into continuous macroscopic fibers. Nanotubes are hollow carbon cylinders that are only one atom thick. Producing fibers for practical use was difficult because nanotubes are chemically complicated. They are strongly attracted to each other and tend to clump in tangled balls. To detangle them, a strong solution of sulfuric acid was used, in much the same way they detangled other strong fibers like Kevlar and Zylon, which was able to disperse up to 10% by weight of pure carbon nanotubes. The acid interacts with the carbon and reassembles the tubes into aligned and mobile fibers. Each strand of the fiber is approximately 100 μm in diameter and contains a million closely packed and aligned nanotubes. Commercially wrought SWNT fibers could have 10 times the tensile strength of Zylon, the strongest fiber currently on the market. Zylon is used by the military and has demonstrated twice the strength of Kevlar. Besides unprecedented strength, the SWNTs are also conductors of electricity and heat. In addition, they can act either as metals or semiconductors. The nanotube fibers are roughly six times lighter than copper.

The application of an electrical charge to single-wall carbon nanotubes produces a direct conversion of electrical energy to mechanical energy through a material response. Using carbon single-wall nanotube sheets, researchers at AlliedSignal Inc. (Morristown, NJ) are in the earliest stages of developing artificial muscle that they believe will be considerably stronger and more durable than human muscle tissue or currently available materials.

The research currently involves the use of a simple nanotube production technique that produces a nanotube “paper.” Described as macroscopic actuators composed of billions of individual nanoscale actuators, these sheets function as a nanotube array in a fashion similar to natural muscle. According to the researchers, “Predictions based on measurements suggest that actuators using optimized nanotube sheets may eventually provide higher work densities per cycle than any previously known technology.” Nanotubes were described as being actually seamless cylinders of graphite. In general, with any type of actuator, there are trade-offs between rate, modulus, and actuator response. There are distinct performance advantages displayed by the use of carbon nanotubes. With polymer-gel actuators, there are generally very-large volume and dimensional changes, but they are slow. They achieve high strokes, but low modulus. The nanotube actuators provide high work density per cycle. While demonstrating a remarkable work capacity per cycle, the nanotube-based muscle requires far less electrical stimulation to function. In terms of required application of power, the energy needed for the nanotube actuator is a full order of magnitude lower than that of polymer gel. By way of illustration, although most thin-film actuators require about 30 V to function, the nanotube material being developed requires approximately 1 V for actuation and about 4 V at most.

It was reported that membranes composed of manmade carbon nanotubes permit a fluid flow nearly 10,000 to 100,000 times faster than fluid flow theory would predict because of the nanotubes'nearly friction-free surface (the Nov. 3, 2005 issue of Nature.) The flow dynamics of carbon nanotube measuring 7 nanometers in diameter permit a fluid flow exceeded the flows predicted by hydrodynamic predictions. These advantages make the aligned carbon nanotubes device a promising large-area platform to enable aqueous flow or viscoelastic flow with little resistance due to boundary effects. Some aspects of the invention provide a method of transporting aqueous by a trabecular stent or viscoelastic by a viscocanalostomy tubing for nearly friction free flow, wherein the stent or tubing is made of carbon nanotubes.

Furthermore, it was reported that water-soluble carbon nanotubes are significantly less toxic (the research finds that nanotubes, like buckyballs, can be rendered nontoxic with minor chemical modifications will be published in an upcoming issue of the journal Toxicology Letters). For medical applications, it is reassuring to see that the cytotoxicity of nanotubes is low and can be further reduced with simple chemical changes (per research report from Rice University). In their native state, carbon nanotubes are insoluble, meaning they are incompatible with the water-based environment of living systems. Solubility is a key issue for medical applications, and researchers at Rice University's Center for Biological and Environmental Nanotechnology (CBEN) and elsewhere have developed processing methods that render nanotubes soluble. Some aspects of the invention provide a trabecular stent made of water-soluble carbon nanotubes.

Cytotoxicity refers to toxic effects on individual cells. In cytotoxicological studies, identical cell cultures are exposed to various forms and concentrations of toxins. In order to compare the toxicity of different compounds, scientists look for the concentration -- typically measured in parts per million or parts per billion -- of materials that lead to the death of 50 percent of the cells in a culture within 48 hours. In the current study, CBEN researchers exposed skin cell cultures to varying doses of four types of water-soluble single-walled carbon nanotubes, or SWNTs. The four included pure, undecorated SWNTs suspended in soapy solution and three forms of nanotubes that were rendered soluble via the attachment of the chemical subgroups hydrogen sulfite, sodium sulfite and carboxylic acid. The cytotoxicity of undecorated SWNTs was 200 parts per billion, which compares to the level of 20 parts per billion identified last year for undecorated buckyballs. The modified nanotubes were non-cytotoxic. While cell death did increase with dose concentration, cell death never exceeded 50 percent for these compounds, which were each tested to a level of 2,000 parts per million. Just as with buckyballs, CBEN found that higher degrees of surface modification led to lower toxicity for SWNTs.

### EXAMPLE NO. 1

### Nano-Coatings

A drug-eluting angioplasty balloon '(manufactured by MILLIMED, Helsingborg, Sweden) delivers a bolus dose of nitric oxide to the angioplastic site during the balloon procedure. It was believed that nitric oxide is a vasodilator and has anti-inflammatory and antiplatelet effects on cells in the vessel wall that may prevent the cell proliferation that can lead to restenosis. The coating on MILLIMED's balloon is made of a polymer into which nitric oxide is incorporated. The polymer is manufactured on a molecular level (nanometer-sized) using an electrical field and is spun into very thin, strong fibers (as small as 3 to 10 molecules in thickness) with enhanced strength, flexibility, porosity and drug-carrying capability.

The balloon coating of a MILLIMED's balloon provides a high bolus dose of nitric oxide at the time of inflation. Although the nitric oxide does not linger in the cells lining the vessel wall, it is believed that the bolus dose changes the cells, making them less likely to initiate an injury response to the balloon inflation.

Some aspects of the present disclosure relate to a method of manufacturing an ophthalmology implant comprising coating at least a portion of the implant with nanometer-sized substances or substrates configured for modifying the physical properties or surface properties of the implant.

Nanocomposites

The term “nanocomposite,” as used herein, generally refers to a composite material comprising a matrix material and a plurality of filler nanometer-sized substance, wherein the filler substance can be smaller than those utilized in filled composites. More particularly, the term “nanocomposites” includes a matrix material comprising a plurality of filler substance (for example, nanoparticles, nanotubes, nanofibers, nano-sheets, nanowires, nanofibrous mesh, and the like) having at least one dimension less than about 100 nm in size. In some embodiments, the filler substance is between about 1 nm and 100 nm. Advantageously, nanocomposite materials can be engineered so that the nanocomposite exhibits the same properties as the matrix material to an enhanced degree and/or exhibits properties in addition to those exhibited by the matrix material alone. Utilizing nanocomposite materials in the manufacture of one or more components of medical devices may allow certain properties of the nanocomposites to be exploited in ways particularly advantageous in the medical device industry.

U.S. Patent Application publication no. 20030093107 published on 5/15/2003, the entire contents of which are incorporated herein by reference, discloses a medical device contemplated to be introduced into the body, either temporarily or permanently, for the purposes of effectuating a treatment or diagnosis thereof in, e.g., urinary, cardiovascular, musculoskeletal, gastrointestinal, or pulmonary applications.

Further, nanocomposite material may comprise property-modifying agents, wherein the agent used would desirably be at least marginally improve the compatibility of the filler particles and the matrix material so that at least a minimal enhancement of the dispersion of the filler particles within the matrix and/or the properties of the nanocomposite can be achieved. Useful amounts of such agents are contemplated to be within the ranges of from about 0.01% to about 10% by weight of the nanocomposite.

In particular, in order to provide a solution of substantially non-aggregated carbon nanotubes that may then be mixed with a similarly dispersed matrix material or simply applied to a matrix material by spraying, coating, or dipping, the carbon nanotubes may be dispersed in an aqueous solution or other organic/inorganic solvents, including natural carbohydrate, starches, gums, and the like. This solution can then be dried to form a substantially non-aggregated powder of carbon nanotubes that may then be compounded with a matrix material and processed into the desired medical device, or the solution may be used to create uniform layers of substantially non-aggregated carbon nanotube fibers on the surface of a matrix material, on the surface of a component of a medical device, or onto substantially the totality of a surface of a medical device. If a uniform layer is desired, once the carbon nanotube solution has been prepared, the desired material may simply be coated with the solution by dipping the material in the solution and allowing the water to evaporate, leaving behind a substantially uniform layer of substantially non-aggregated carbon nanotubes.

Such a layer of carbon nanotubes may be used as a tie layer between polymer layers of a medical device, for example, by depositing the carbon nanotubes as described on at least one of the surfaces to be thermally bonded. Upon thermal bonding of the two layers, the interspersed tie layer of carbon nanotubes would provide additional reinforcement to the bondsite or enhanced conductivity. This advantageous technology may be applied to embodiments where a tie layer is desired between two layers of material wherein the second layer of material is applied to the first via welding, spraying, or multilayer extrusion and/or wherein electrical conductivity is desired. In such embodiments, the carbon nanotube solution can simply be applied to the first material and allowed to dry, and the second material subsequently applied according to the desired technology over the substantially uniform carbon nanotube layer.

Generally, one of the advantages of the utilization of nanocomposites is that, at least as compared to traditionally filled polymers, nanocomposites are often more easily processed. As a result, once the nanocomposite has been prepared, it can be processed into the desired medical device by any method known to those of ordinary skill in the art, and the particular method chosen is not critical to the practice of the present disclosure. There are a multiplicity of methods for the manufacture of medical devices that are thus appropriate, examples of which include, but are not limited to, foam processing, blow molding or film molding, sheet forming processes, profile extrusion, rotational molding, compression molding, thermoset pre-preg processes and reaction injection molding processes.

PCT WO 99/18893, the entirety of which is hereby incorporated by reference, describes a method for preparing nanofibrils from both nondegrading and biodegradable polymers for use as tissue engineering scaffolds. Further, U.S. Patent Application publication no. 20030021821 published on 1/30/03, entire contents of which are incorporated herein by reference, discloses collagen or collagen-like material (could be contemplated as a property-modifying agent or a matrix material) that is incorporated into nanometer-sized substance or substrate of the present disclosure as illustrated in the following examples so that others skilled in the art may appreciate and understand the principles and practices of the present disclosure.

### EXAMPLE NO. 2

Synthesis of 3-dimensional Nanofibrous Matrices Containing Recombinant Collagen: Nanofibrillar matrices were synthesized using polymers with free NH₂ groups for the covalent binding of collagen (Zheng et al. In Vitro Cell Devel. Biol. Anim. 1998 34:679-84). Specifically, poly(L-lactic acid) (MW 200,000; Polysciences, Inc) was mixed with poly(ε-CBZ-L-lysine) (MW 260,000; Sigrna) at a 4:1 ratio. The carbobenzoxy (CBZ)-protected form of L-lysine was used to prevent involvement of side chain groups in the formation of a CONH bond during peptide synthesis. A mixture of polymers was then dissolved in chloroform and used to generate nanofibrillar material in the electrostatic spinning process. In this nonmechanical technique, a high electric field is generated between a polymer fluid contained in a glass syringe with a capillary tip and a metallic collection screen. When the voltage reaches a critical value, the charge overcomes the surface tension of the deformed drop of the suspended polymer solution created on the capillary tip, producing a jet. The electrically charged jet undergoes a series of electrically induced bending instabilities during its passage to the collection screen, hyperstretching the jet. This process is accompanied by the rapid evaporation of the solvent. The dry fibers are accumulated on the surface of the collection screen, resulting in a nonwoven mesh of nanofibers. The covalent binding of the collagen was done according to the method developed by Zheng et al. To activate CBZ-protected ε-amino groups, the matrices were placed in a 4.5M HCl solution in glacial acetic acid and incubated for 30 minutes at 37° C. The samples were neutralized by the addition of 0.1M sodium carbonate and then stored in sterile water at 4° C. Recombinant collagen stock solutions were diluted to a final concentration of 200 μg/mL with 10 mM of MOPS [3-(N-Morpholino) propanesulfonic acid], adjusted to pH 4.5, containing 5 mg/mL of water-soluble carbodiimide [1-ethyl-3-(3-bimethylam- inopropyl) carbodiimide; Pierce]. The activated amino groups were permitted to react with collagen for 48 hours at 4° C. Unbound collagen was then removed by washing of the matrices with 10 mM of HCl, followed by a washing with water. The efficiency of incorporation of collagen into nanofibrous matrices was determined by an analysis of the hydroxyproline content after acid hydrolysis and reaction with p-dimethylaminobenzaldehyde.

Electrical Conductivity

A medical device can include a plurality of conductive nanometer-sized substances disposed on at least a portion of the device, wherein the substances are selected from the group consisting of a carbon nanotube material, a boron nanotube material, a carbon nanowire material, a carbon nanofibrill material, a doped nanotube material, and an electrically modified nanotube material.

Carbon nanotubes can be formed to have metallic conductor or semi-conductor properties and are capable of transmitting electrical or neural signals. Carbon nanotubes are thin, long tubular macromolecules with diameters on the order of a 1-200 nanometers (molecules are on the order of a few nanometers) and with lengths on the order of micrometers to millimeters. Bundles of such nanotubes create nanostructures that are characterized by a large surface area. In short, these characteristics of carbon nanotubes may make them particularly well-suited for diverse uses in conjunction with neural medical implant for improving electrical or neural signal performance.

IOP Sensors

It is contemplated that an innovative nanometer-sized device could possibly be sized and configured for identifying and/or killing cancer cells. The device would conceptually have a small computer, several binding sites to determine the concentration of specific molecules, and a supply of some poison which could be selectively released and was able to kill a cell identified as cancerous. The device would circulate freely throughout the body, and would periodically sample its environment by determining whether the binding sites were or were not occupied. Occupancy statistics would allow determination of concentration. The cancer-killing device suggested here could incorporate a dozen different binding sites and so could conceptually monitor the concentrations of a dozen different types of molecules. The computer could determine if the profile of concentrations fit a pre-programmed “cancerous” profile and would, when a cancerous profile was encountered, release the poison.

Beyond being able to determine the concentrations of different compounds, the cancer-killer or a nanometer-sized intraocular pressure sensor could also determine local pressures. A pressure sensor or nanosensor element little more than 10 nanometers on a side would be sufficient to detect pressure changes of less than 0.1 atmospheres. As acoustic signals in the megahertz range are commonly employed in diagnostics (ultrasound imaging of pregnant women, for example), the ability to detect such signals would permit the nanopressure sensor to safely receive broadcast instructions. By using several macroscopic acoustic signal sources, the nanopressure sensor could determine its location within the body much as a radio receiver on earth can use the transmissions from several satellites to determine its position (as in the widely used GPS system). Megahertz transmission frequencies would also permit multiple samples of the pressure to be taken from the pressure sensor, as the CPU would be operating at gigahertz frequencies. An intraocular pressure sensor can include nanosensor elements that are sized and configured less than 100 nanometers in at least one dimension.

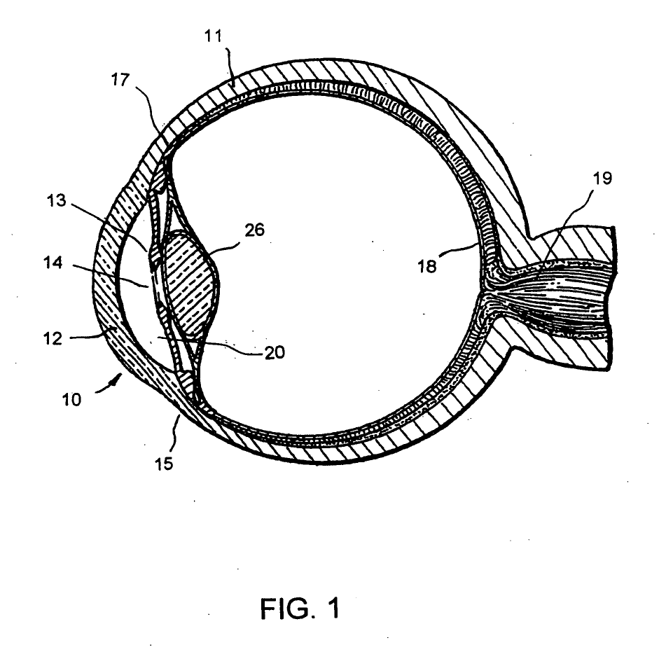

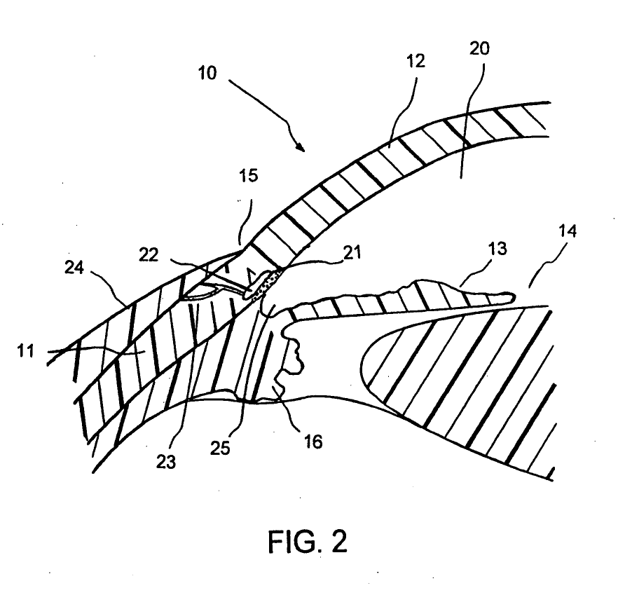

FIG. 1 shows a cross-sectional view of an eye 10, while FIG. 2 shows a close-up view showing the relative anatomical locations of a trabecular meshwork 21, an anterior chamber 20, and a Schlemm's canal 22. A sclera 11 is a thick collagenous tissue that covers the entire eye 10 except a portion which is covered by a cornea 12. The cornea 12 is a thin transparent tissue that focuses and transmits light into the eye and through a pupil 14, which is a circular hole in the center of an iris 13 (colored portion of the eye). The cornea 12 merges into the sclera 11 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

The anterior chamber 20 of the eye 10, which is bound anteriorly by the cornea 12 and posteriorly by the iris 13 and a lens 26, is filled with aqueous humor (hereinafter referred to as “aqueous” ). Aqueous is produced primarily by the ciliary body 16, then moves anteriorly through the pupil 14 and reaches the anterior chamber angle 25, formed between the iris 13 and the cornea 12. In a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20 which leads to an increase in intraocular pressure. Fluids are relatively incompressible, and thus intraocular pressure is distributed relatively uniformly throughout the eye 10.

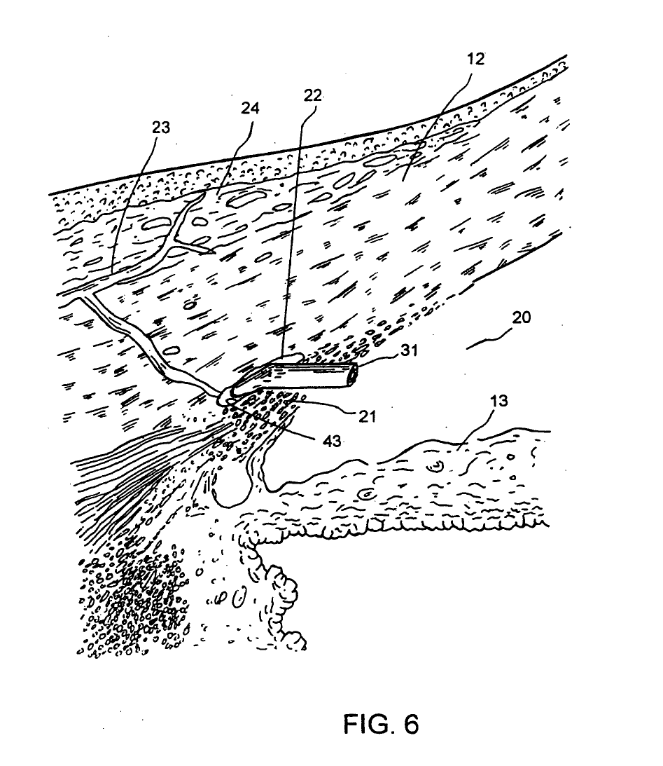

As shown in FIG. 2, the trabecular meshwork 21 is adjacent to a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12. In one embodiment, a trabecular stent 31 is placed bypassing the trabecular meshwork 21 with a distal opening disposed (or exposed) in Schlemm's canal 22 and a proximal opening disposed (or exposed) in the anterior chamber 20 as illustrated in FIG. 6.

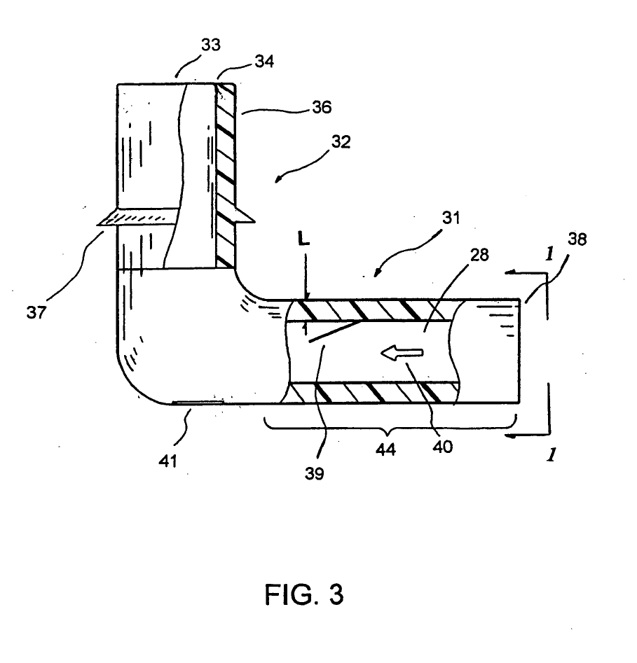

FIG. 3 shows an embodiment of the trabecular stent implant 31 constructed according to the principles of the disclosure. The trabecular implant may comprise a biocompatible material, such as a medical grade silicone, for example, the material sold under the trademark SILASTIC™, which is available from Dow Corning Corporation of Midland, Mich., or polyurethane, which is sold under the trademark PELLETHANE™, which is also available from Dow Corning Corporation. In an alternate embodiment, other biocompatible materials (biomaterials) may be used, such as polyvinyl alcohol, polyvinyl pyrolidone, collagen, heparinized collagen, tetrafluoroethylene, fluorinated polymer, fluorinated elastomer, flexible fused silica, titanium, stainless steel, polyolefin, polyester, polysilicon, silicone, polyurethane, mixture of biocompatible materials, and the like. In a further alternate embodiment, a composite biocompatible material by surface coating the above-mentioned biomaterial may be used, wherein the coating material may be selected from the group consisting of polytetrafluoroethylene (PTFE), polyimide, hydrogel, heparin, therapeutic drugs, and the like.

The main purpose of the trabecular implant is to assist in facilitating the outflow of aqueous in an outward direction 40 into the Schlemm's canal and subsequently into the aqueous collectors and the aqueous veins so that the intraocular pressure is balanced. In one embodiment, the trabecular implant 31 comprises an elongated tubular element having a distal section 32 and an inlet section 44. A rigid or flexible distal or outlet section 32 is positioned inside one of the existing outflow pathways. The distal section may have either a tapered outlet end 33 or have at least one ridge 37 or other retention device protruding radially outwardly for stabilizing the trabecular implant inside the existing outflow pathways after implantation. For stabilization purposes, the outer surface of the distal section 32 may comprise a stubbed surface, a ribbed surface, a surface with pillars, a textured surface, or the like. The outer surface 36 and the inner region 34 at the outlet end 33, of the trabecular implant is biocompatible and tissue compatible so that the interaction/irritation between the outer surface and the surrounding tissue is minimized. The trabecular implant may comprise at least one opening at a location proximal the distal section 32, away from the outlet end 33, to allow flow of aqueous in more than one direction. The at least one opening may be located on the distal section 32 at about opposite of the outlet end 33. In one embodiment, the trabecular stent is an “L” shaped, “I” shaped, or “T” shaped device.

In another exemplary embodiment, the trabecular implant 31 may have a one-way flow controlling means 39 for allowing one-way aqueous flow 40. The one-way flow controlling means 39 may be selected from the group consisting of a check valve, a slit valve, a micropump, a semi-permeable membrane, or the like. To enhance the outflow efficiency, at least one optional opening 41 in the proximal portion of the distal section 32, at a location away from the outlet end 33, and in an exemplary embodiment at the opposite end of the outlet end 33, may be provided.

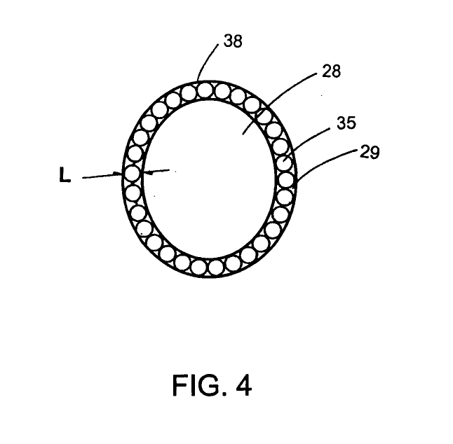

In one embodiment, at least a portion of the trabecular stent 31 or 45 may comprise nanometer-sized nanotubes, such as carbon nanotubes configured in essentially parallel to aqueous flow inside the lumen 28. In another embodiment, at least a portion of the trabecular stent may comprise nanometer-sized substance, such as nanotubes, nanofibers, nanowires, nanofibrous mesh and combination thereof. FIG. 4 shows a front cross-sectional view of the proximal end 38 of FIG. 3. The shape of the openings of the inlet end 33, the outlet end (not shown) and the remaining body of the device 31 may be oval, round, or some other shape adapted to conform to the shape of the existing outflow pathways. The configuration of the outlet section would match the contour of Schlemm's canal to stabilize the outlet section within the canal. In one embodiment as shown in FIG. 4, the trabecular stent comprises a plurality of carbon nanotubes 35 and a matrix material 29 to hold the nanotubes together. Since the nanotubes have a tensile strength 100 times that of steel at only one-sixth the weight, it is feasible to have only one or a few layers of nanotubes for device construction. The device thickness L can be as small as a few nanometers to maximize the luminal aqueous flow cross-sectional area, as compared to a stent with a wall thickness in the order of microns. Some aspects of the disclosure is to provide a trabecular stent or a tubular medical device sized and configured with a wall thickness less than about 100 nanometers, preferably less than about 10 nanometers, more preferably less than about 2 nanometers.

A medical device for ophthalmology implantation can have at least a portion of the device including a nanostructure. In a further embodiment, the nanostructure is associated with nanometer-sized substance. In a further embodiment, the nanometer-sized substance is selected from the group consisting carbon nanotubes, nanofibers, nanowires, and nanofibrous mesh. “Nanostructure” is intended herein to mean either or both of (a) the macroscopic structure of a medical device made of or incorporated with nanometer- or sub-nanometer-sized substances; or (b) the microscopic structure of a nanoparticle, nanosheet, nantoube, nanowire, nanomesh, or the like.

As shown in FIG. 3, the trabecular implant of the present disclosure may have a length between about 0.5 mm to over a meter, depending on the body cavity the trabecular implant applies to. The outside diameter of the trabecular implant may range from about 1 μm to about 500 μm, preferably from about 10 μm to about 100 μm. The lumen diameter is preferably in the range between about 1 μm to about 150 μm, preferably from about 10 μm to about 100 μm. With the extremely high tensile strength of carbon nanotubes, a trabecular stent or other tubular medical device comprised of nanotubes may show a ratio of wall thickness to inside diameter as less than about one-hundredth, preferably less than about one-thousandth, more preferably less than about one-ten thousandth, and most preferably less than about one-millionth. The trabecular implant may have a plurality of lumens to facilitate multiple flow transportation. The distal section may be curved at an angle between about 30 degrees to about 150 degrees, in an exemplary embodiment at around 70-110 degrees, with reference to the inlet section 44.

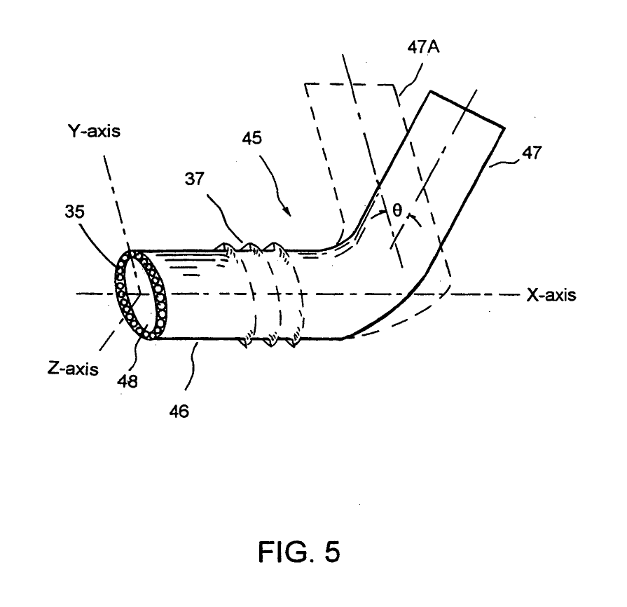

FIG. 5 shows another embodiment of the trabecular implant 45 constructed in accordance with the principles of the disclosure. In an exemplary embodiment, the trabecular implant 45 may comprise at least two sections: an inlet section 47 and an outlet section 46. The outlet section has an outlet opening 48 that is at the outlet end of the trabecular implant 45. The shape of the outlet opening 48 is preferably an oval shape to conform to the contour of the existing outflow pathways. A portion of the inlet section 47 adjacent the joint region to the outlet section 46 will be positioned essentially through the diseased trabecular meshwork while the remainder of the inlet section 47 and the outlet section 46 are outside the trabecular meshwork. In one preferred embodiment, the outlet section 46 is coextensive with the inlet section 46 axially. As shown in FIG. 5, the long axis of the oval shape opening 48 lies in a first plane formed by an X-axis and a Y-axis. To better conform to the anatomical contour of the anterior chamber 20, the trabecular meshwork 21 and the existing outflow pathways, the inlet section 47 may preferably lie at an elevated second plane, at an angle θ, from the first plane formed by an imaginary inlet section 47A and the outlet section 46. The angle θ may be between about 30 degrees and about 150 degrees. In one embodiment, at least a portion of the trabecular stent 45 comprises nanometer-sized nanotubes or other nanometer-sized substance.

One aspect of the disclosure includes a method for increasing aqueous humor outflow in an eye of a patient, to reduce the intraocular pressure therein. The method comprises bypassing the trabecular meshwork 21. The device 31 may be elongate or of other appropriate shape, size, or configuration, as will be evident to those of skill in the art. The method includes the following: (a) creating an opening in the trabecular meshwork 21, wherein the trabecular meshwork 21 includes a deep side and superficial side; (b) inserting a glaucoma device or a trabecular stent into the opening; and (c) transmitting aqueous humor through the device, to bypass the trabecular meshwork 21. This “transmitting” of aqueous humor is, in one aspect of the disclosure, preferably passive, i.e., aqueous humor is allowed to flow out of the anterior chamber due to the pressure gradient between the anterior chamber and the aqueous venous system.

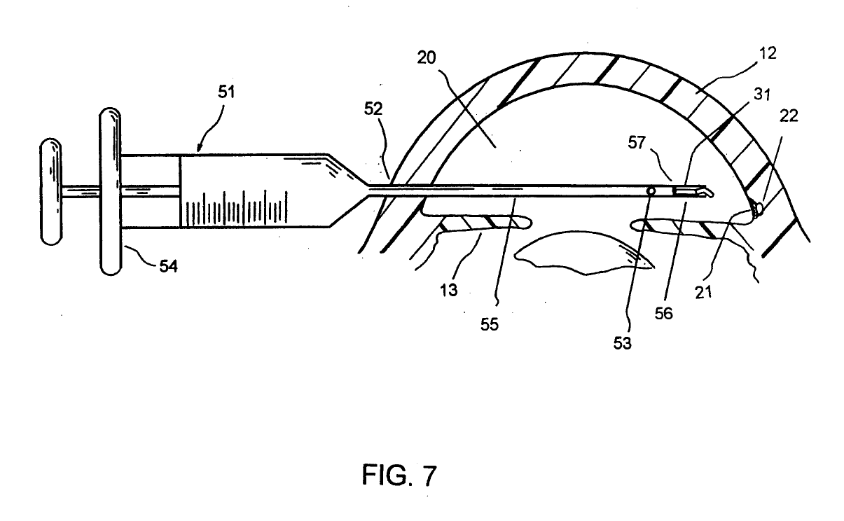

FIG. 7 shows an aspect of placing the glaucoma device or a trabecular stent at the implantation site. An irrigating knife, stent delivery apparatus, or applicator 51 is provided, which, in some embodiments, comprises a syringe portion 54 and a cannula portion 55. In one embodiment, the distal section of the cannula portion 55 has at least one irrigating hole 53 and a distal space 56 for holding the device 31. The proximal end 57 of the lumen of the distal space 56 is, in one embodiment, sealed off from, and thus substantially not in communication with, the remaining lumen of the cannula portion 55. In this embodiment, the device is placed on the delivery applicator and advanced to the device site, wherein the delivery applicator holds the device securely during delivery and releases it when the surgeon chooses to deploy the device. In one embodiment, the delivery apparatus holds more than trabecular stent for multiple delivery operations.

For positioning the trabecular stent 31 in the slit, hole or opening through the trabecular meshwork, the trabecular stent may be advanced over the guidewire or a fiberoptic (retrograde). In another embodiment, the trabecular stent is directly placed on the delivery applicator and advanced to the implant site, wherein the delivery applicator holds the trabecular stent securely during the delivery stage and releases it during the deployment stage.

In some embodiments of trabecular meshwork surgery in accordance with the disclosure, the patient is placed in the supine position, prepped, draped, and anesthetized as necessary. In one embodiment, a small (less than about 1-mm) incision, which may be self-sealing, is made through the cornea. Through this incision, the trabecular meshwork 21 is accessed, and an incision is made in the trabecular meshwork 21 with an irrigating knife. The device, 31 or 45, is then advanced through the corneal incision 52 across the anterior chamber 20, while the device is held in an irrigating applicator 51, under gonioscopic, microscopic, or endoscopic guidance. After the device is implanted in place, the applicator is withdrawn and the surgery concluded. The irrigating knife may be within a size range of about 16 to about 40 gauges, and, in some embodiments, preferably about 30 gauges.

In some embodiments, other shapes of implants and delivery devices may be used for disposing the stent within the trabecular meshwork of the eye. For example, several embodiments are disclosed in U.S. patent application Ser. No. 11/083,713, filed Mar. 18, 2005, the entire contents of which are hereby incorporated by reference. For example, the implant can have a bulbous portion or have a mushroom shape, as described in the application referenced above.

FIG. 6 illustrates the device 31 positioned within the tissue of an eye 10. An opening is present in the trabecular meshwork 21. The outlet section 32 of the device 31 has been inserted into the opening. The inlet section 44 is exposed to the anterior chamber 20, while the outlet section is positioned near an interior surface 43 of the trabecular meshwork 21. In a further embodiment, the outlet section may further be placed into fluid collection channels, as described above.

In one embodiment, the method of forming an opening in the trabecular meshwork 21 may comprise making an incision with a microknife, a pointed guidewire, a sharpened applicator, a screw-shaped applicator, an irrigating applicator, or a barbed applicator. Alternatively, the trabecular meshwork 21 may be dissected with an instrument similar to a retinal pick or microcurrette. The opening may alternately be created by fiberoptic laser ablation.

Stent With Telescoping Antenna

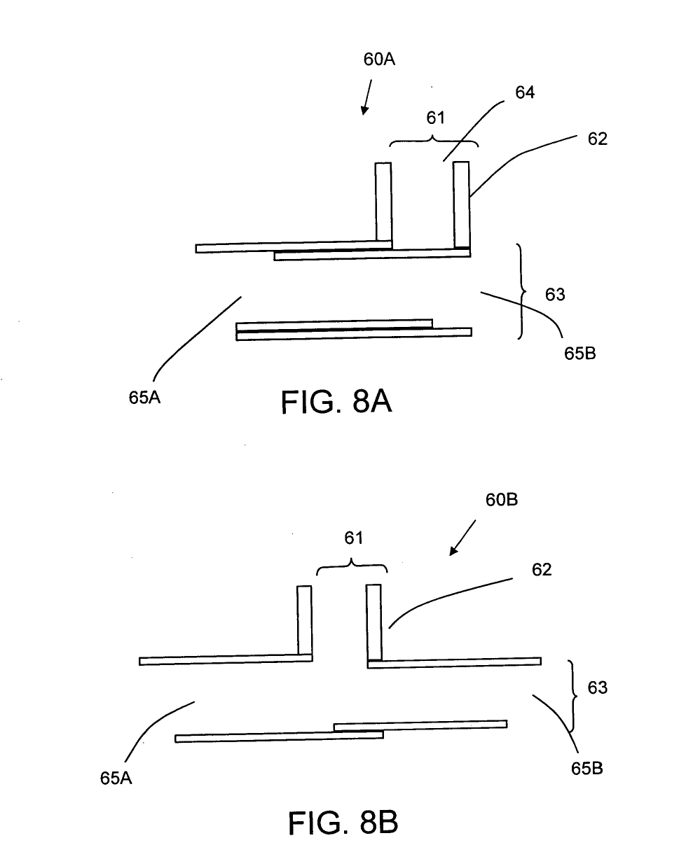

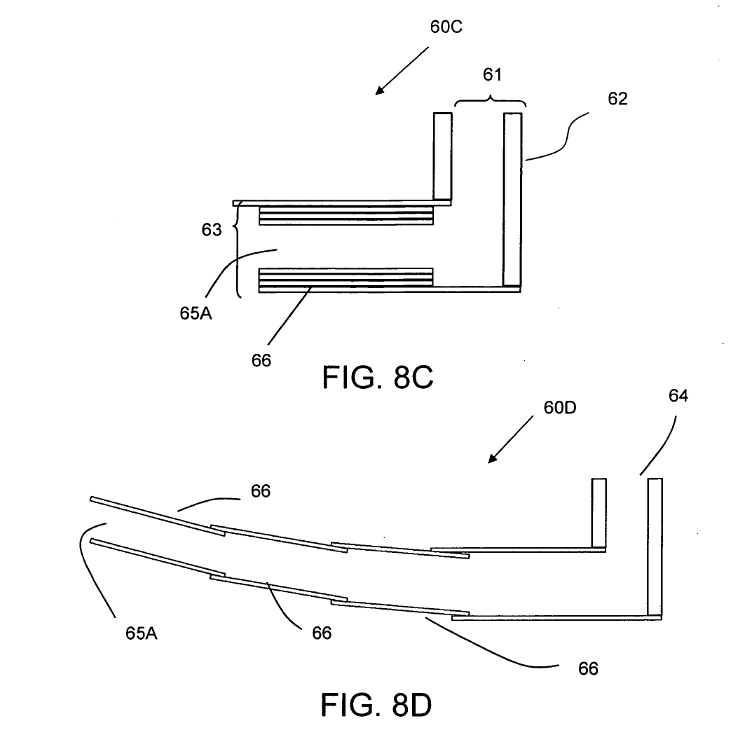

A trabecular bypass stent 60A with wall material made with nano-sized material is sized and configured in L-shape geometry, as shown in FIG. 8A. The stent comprises an inlet opening 64 at an inlet section 61 and at least one outlet opening 65A, 65B at an outlet section 63, wherein the wall 62 may comprise carbon nanotubes, sheets from carbon nanotubes, or woven tubing made of nanotubes. The inlet section is also known as the snorkel or telescoping antenna of the device. In one alternate embodiment, a portion of the outlet section 63 is extended out in one direction to form a T-shaped stent 60B from its original L-shaped geometry, as shown in FIG. 8B, with adjustable anchoring capability (axially extendable) within Schlemm's canal. In general, the inlet opening is placed in the anterior chamber while the outlet opening is placed within Schlemm's canal. Other shapes may also be used. For example, the stent may include a cylindrical shape with outlets openings along the sides of the stent.

In another embodiment, the L-shaped stent 60C with one outlet opening 65A may contain at least one inner tube 66 (showing three inner tubes in FIG. 8C). In a further embodiment, the inner tubes of the stent 60D are deployable in Schlemm's canal after implantation in one direction (FIG. 8D) or in both directions (not shown). With many short segments of the inner tubes, the deployed stent 60D can have a curvature conforming to the nature of Schlemm's canal configuration. The cross-sectional shape of the stent in Schlemm's canal can be circular, rectangular, or ideally oval, with close tube or open tube (that is exposed to the collector channels) designs. The trabecular stent can be made of nano-sized material that have an adjustable anchoring capability with Schlemm's canal.

Although preferred embodiments of the disclosure have been described in detail, including implant comprised of nanometer-sized substance or nanostructure, certain variations and modifications will be apparent to those skilled in the art, including embodiments that do not provide all of the features and benefits described herein. Accordingly, the scope of the present disclosure is not to be limited by the illustrations or the foregoing descriptions thereof, but rather solely by reference to the appended claims.

What is claimed:

1. An implant for treating glaucoma comprising:
an inlet portion having an inlet lumen extending therethrough, the inlet lumen being in fluid communication with the anterior chamber of an eye when the implant is positioned within the trabecular meshwork of the eye; and
an outlet portion having an outlet lumen extending therethrough, the outlet lumen being in fluid communication with the inlet lumen and with an aqueous outflow channel of the eye when the outlet portion is disposed within the aqueous outflow channel of the eye and thereby permitting the transfer of fluid from the anterior chamber into the aqueous outflow channel;
wherein at least a portion of the implant comprises a nanostructure; and
wherein the aqueous outflow channel comprises at least one of Schlemm's canal, a collector channel, and an episcleral vein.

2. The implant of claim 1, wherein the nanostructure comprises at least one of a nanotube, a nanofiber, a sheet of nanotubes, a nanowire, and a nanomesh.

3. The implant of claim 1, wherein the nanostructure comprises a nanotube.

4. The implant of claim 1, wherein the nanostructure comprises a nanofiber.

5. The implant of claim 1, wherein the nanostructure comprises a sheet of nanotubes.

6. The implant of claim 1, wherein the nanostructure comprises a nanomesh.

7. The implant of claim 1, wherein the nanostructure comprises a carbon nanotube.

8. The implant of claim 1, wherein the nanostructure comprises a carbon nanofiber.

9. The implant of claim 1, wherein the nanostructure comprises a single-wall carbon nanostructure.

10. The implant of claim 1, wherein the nanostructure comprises a multi-wall carbon nanostructure.

11. The implant of claim 1, wherein a long axis of the nanostructure is aligned substantially parallel to the flow of fluid through one of the lumens of the stent.

12. The implant of claim 1, wherein the nanostructure is electrically conductive.

13. The implant of claim 1, wherein the inlet lumen and the outlet lumen are substantially parallel.

14. The implant of claim 1, wherein at least one of the inlet portion and the outlet portion comprises a wall having at least a portion thereof comprising a nanostructure.

15. The implant of claim 14, wherein the wall has a wall thickness less than about 100 nanometers.

16. The implant of claim 1, wherein the implant further comprises means for detecting intraocular pressure.

17. The implant of claim 1, wherein the implant further comprises an intraocular pressure sensor.

18. The implant of claim 1, wherein the implant further comprises a therapeutic agent.

19. The implant of claim 1, wherein the implant further comprises means for anchoring the implant in the eye.

20. The implant of claim 1, wherein the implant further comprises an anchor that substantially prevents expulsion of the implant from the eye.

21. The implant of claim 1, wherein the implant is substantially linear prior to insertion in the eye.

22. An implant for treating glaucoma comprising:
an inlet portion having an inlet that is configured to receive fluid from the anterior chamber of an eye when the implant is positioned within the trabecular meshwork of the eye;
an outlet portion having an outlet that is configured to conduct fluid from the anterior chamber into at least one of Schlemm's canal, a collector channel, and an episcleral vein of the eye when the outlet portion is positioned within the eye, thereby permitting the transfer of fluid from the anterior chamber into that at least one of Schlemm's canal, a collector channel, and an episcleral veins; and
a layer of nanotubes disposed in or on the implant.

23. The implant of claim 22, wherein the layer of nanotubes is disposed in or on an interior portion of the implant.

24. The implant of claim 22, wherein the layer of nanotubes is disposed in or on the implant by spraying a matrix comprising the nanotubes on the implant.

25. The implant of claim 22, wherein the layer of nanotubes is disposed in or on the implant by dipping the implant into a matrix comprising the nanotubes.

26. The implant of claim 22, wherein the implant further comprises a therapeutic agent.

27. The implant of claim 22, wherein the implant is substantially I-shaped.

28. The implant of claim 22, wherein the implant is substantially linear prior to insertion in the eye.

29. The implant of 22, wherein the implant further comprises an anchor that substantially prevents expulsion of the implant from the eye.
